(19) United States
(12) Patent Application Publication
(10) Pub. No.: US 2026/0345979 A1
(43) Pub. Date: Nov. 5, 2026

(54) MICRONEEDLE FILM AND MANUFACTURING METHOD THEREOF

(71) Applicant: DARWIN PRECISIONS CORPORATION, Hsinchu County (TW)

(72) Inventors: WEI-TANG CHENG, Hsinchu County (TW); Yun-Pei Yang, Hsinchu County (TW)

(21) Appl. No.: 19/570,092

(22) Filed: Mar. 17, 2026

(30) Foreign Application Priority Data
May 5, 2025 (TW) 114116790

(51) Int. Cl. A61M 37/00

(52) U.S. Cl. CPC A61M 37/0015; A61M 2037/0046; A61M 2037/0053

(57) ABSTRACT

A microneedle film includes a base layer, a plurality of microneedles, and a plurality of microstructures. The base layer has a first surface and a second surface opposite to the first surface, and the first surface is rougher than the second surface. The microneedles are disposed on the first surface of the base layer and spaced apart from each other. Each of the microstructures corresponds to a respective one of the microneedles. Each of the microstructures includes a plurality of grooves, and each of the grooves forms a pattern of a geometric shape or a line segment on the first surface. The line segment includes a straight line or a curve line.

### FIELD OF THE INVENTION

The present invention relates to the field of transdermal drug delivery, and more particularly to a microneedle film and a manufacturing method thereof.

### BACKGROUND OF THE INVENTION

Microneedle patches (MNPs) are a novel type of transdermal drug delivery system (TDDS). The microneedles on the patch are extremely short and do not reach the nerve endings, thereby avoiding the pain associated with subcutaneous injections. In addition, the microneedles are capable of delivering bioactive agents or pharmaceutical compounds through the stratum corneum into the human body. MNP technology can be applied in various fields, including aesthetic medicine, pharmaceuticals, and preventive healthcare.

### SUMMARY OF THE INVENTION

The present invention provides a microneedle film with a high molding yield.

The present invention further provides a method of manufacturing a microneedle film, which facilitates improvement in the molding yield of the microneedle film.

To achieve one, some, or all of the aforementioned purposes or other purposes, an embodiment of the present invention provides a microneedle film including a base layer, a plurality of microneedles, and a plurality of microstructures. The base layer includes a first surface and a second surface opposite to the first surface. The first surface is rougher than the second surface. The microneedles are disposed on the base layer. The microneedles are spaced apart from each other and distributed on the first surface of the base layer. The microstructures are formed on the first surface. Each of the microstructures corresponds to a respective one of the microneedles. Each of the microstructures includes a plurality of grooves. Each of the grooves forms a geometric shape or a line segment on the first surface. The line segment includes a straight line or a curved line.

In an embodiment of the present invention, the groove has a width between 0.975 μm and 120 μm and a depth between 0.975 μm and 10 μm. A spacing between the adjacent grooves is between 0 and 120 μm.

According to an embodiment of the present invention, a method of manufacturing a microneedle film is also provided. The method first provides a first mold. The first mold includes a support base and a plurality of microneedle structures. The microneedle structures are spaced apart from each other and disposed on the support base. A plurality of micro-groove structures are formed on the surface of the support base. Each of the micro-groove structures corresponds to a respective one of the microneedle structures. Each of the micro-groove structures includes a plurality of groove structures. Next, a polymer material is provided into the first mold to form a second mold. After that, a microneedle material is provided to the second mold to form a microneedle film. In the step of forming the microneedle film, it further includes forming a plurality of microneedles corresponding to the microneedle structures on the microneedle film, forming a first surface of the microneedle film corresponding to the surface of the support base, on which the microneedles are spaced apart from each other and distributed, and forming a plurality of microstructures corresponding to the micro-groove structures. Each of the microstructures includes a plurality of grooves.

The present invention employs the microstructures to enhance the adhesion between the microneedle film material and the mold, thereby facilitating improvement in the molding yield of the microneedle film. As a result, the film formation quality is enhanced, the microneedle structures are intact, and the production yield is increased.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic side view of a microneedle film according to a first embodiment of the present invention.

FIG. 2 is a partial schematic top view of the microneedle film in FIG. 1.

FIG. 3 is a partial cross-sectional schematic view of the microneedle film taken along A′A′ in FIG. 2.

FIG. 4A is a partial cross-sectional schematic view of a microneedle film according to a second embodiment of the present invention.

FIG. 4B is a partial cross-sectional schematic view of a microneedle film according to a third embodiment of the present invention.

FIG. 4C is a partial cross-sectional schematic view of a microneedle film according to a fourth embodiment of the present invention.

FIG. 4D is a partial cross-sectional schematic view of a microneedle film according to a fifth embodiment of the present invention.

FIG. 5A is a partial schematic top view of a microneedle film according to a sixth embodiment of the present invention.

FIG. 5B is a partial schematic top view of a microneedle film according to a seventh embodiment of the present invention.

FIG. 5C is a partially enlarged schematic diagram of the microneedle film in FIG. 5B.

FIG. 5D is a partial schematic top view of a microneedle film according to an eighth embodiment of the present invention.

FIG. 5E is a partial schematic top view of a microneedle film according to a ninth embodiment of the present invention.

FIG. 5F is a partially enlarged schematic diagram of the microneedle film in FIG. 5E.

FIG. 5G is a partial schematic top view of a microneedle film according to a tenth embodiment of the present invention.

FIG. 5H is a partial schematic top view of a microneedle film according to an eleventh embodiment of the present invention.

FIG. 5I is a partial schematic top view of a microneedle film according to a twelfth embodiment of the present invention.

FIG. 6A is a schematic flow chart of a method of manufacturing a microneedle film according to an embodiment of the present invention.

FIG. 6B is a schematic flow chart of a method of manufacturing a microneedle film according to another embodiment of the present invention.

FIG. 7A is a schematic diagram of a method of manufacturing a microneedle film according to an embodiment of the present invention.

FIG. 7B is a schematic diagram of a method of manufacturing a microneedle film according to another embodiment of the present invention.

### DETAILED DESCRIPTION OF PREFERRED EMBODIMENTS

In the following description of embodiments according to the present invention, terms indicating orientation or positional relationships, such as “upper,” “lower,” etc., are described based on the orientation or positional relationships shown in the corresponding drawings. These terms are used solely for the convenience of describing the invention and are not intended to limit the invention or to imply that the referenced elements must have a particular orientation or be constructed in a particular orientation. Furthermore, the terms “first,” “second,” and the like, as used in this specification or the claims, are employed merely to designate names of elements or to distinguish between different embodiments or ranges and are not intended to limit the number of elements.

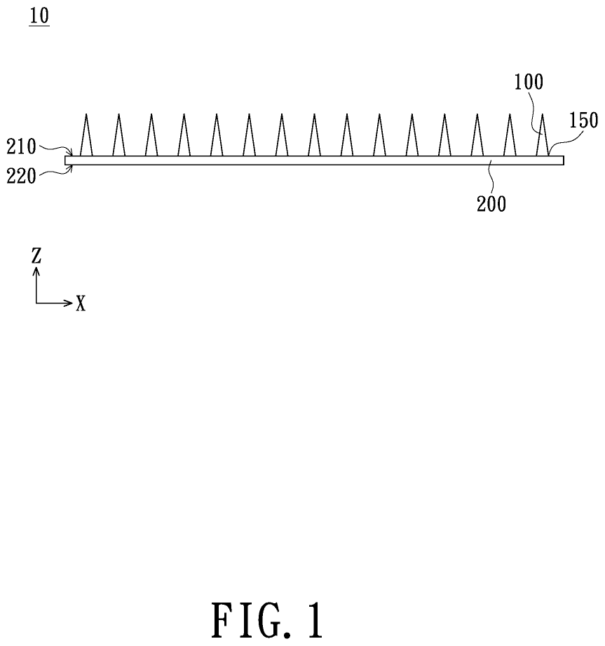

The present invention provides a microneedle film which can be used to form a microneedle patch for transdermal drug delivery. FIG. 1 is a schematic side view of a microneedle film according to a first embodiment of the present invention. As shown in FIG. 1, a microneedle film 10 includes a plurality of microneedles 100 and a base layer 200. The microneedles 100 are disposed on the base layer 200. Each of the microneedles 100 includes a bottom 150 which is connected to the base layer 200. A shape of the microneedle 100 may be conical, for example, a circular cone, triangular pyramid, or quadrangular pyramid. The microneedle 100 gradually tapers upward from the wider base 150. A vertical distance from the base 150 to a top of the microneedle 100 corresponds to a height of the microneedle 100. The height of the microneedle 100 may be, for example, between 150 μm and 1000 μm.

The base layer 200 includes a first surface 210 and a second surface 220 opposite to the first surface 210. Preferably, the first surface 210 and the second surface 220 have different roughness. For example, one of the two surfaces may be a rough surface having certain roughness while the other surface is a smooth surface. Alternatively, one of the two surfaces is rougher than the other surface. In the embodiment shown in FIG. 1, the first surface 210 is rougher than the second surface 220. The microneedles 100 are spaced apart from each other and distributed on the first surface 210 of the base layer 200. The second surface 220 may be regarded as a bottom surface of the microneedle film 10. A spacing between the adjacent microneedles 100 may be, for example, between 0.5 mm and 5 mm, including 0.5 mm, 1.0 mm, 2.0 mm, 2.5 mm, 3.0 mm, 3.5 mm, 4.0 mm, 4.5 mm, and 5 mm. However, the invention is not limited thereto. Preferably, the microneedles 100 are distributed uniformly on the first surface 210. In some embodiments, the microneedles 100 may be arranged in rows or columns and distributed uniformly on the first surface 210, though the invention is not limited thereto. For example, the microneedles 100 may be randomly distributed on the first surface 210 to achieve the uniform distribution effect. A carrier layer (as described later) may be disposed on the bottom surface of the microneedle film 10 to form the microneedle patch. A material of the carrier layer may be different from a material of the microneedle film 10. The carrier layer may include, for example, a natural or synthetic material, such as non-woven fabric, cotton cloth, a polyester film, or a plastic film, which may provide a structural support and make the microneedle film 10 or the microneedle patch suitable for operating and applying to a body surface.

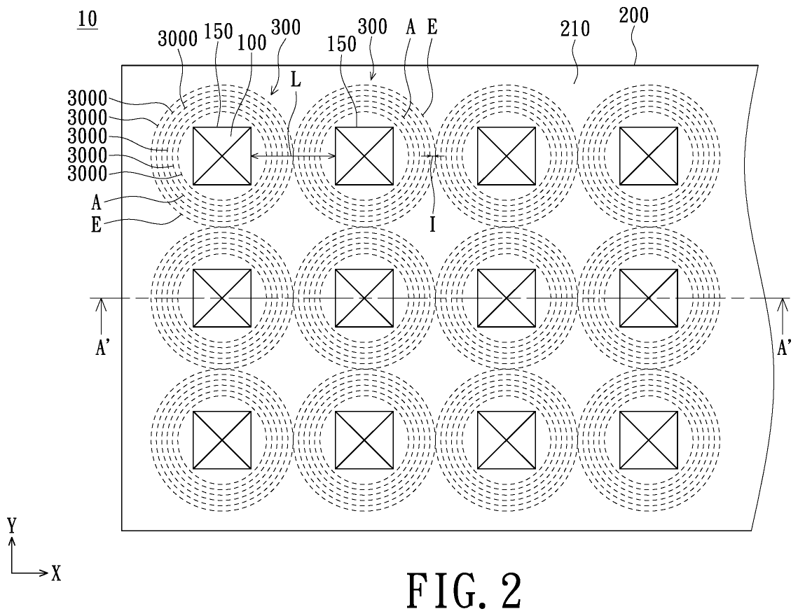

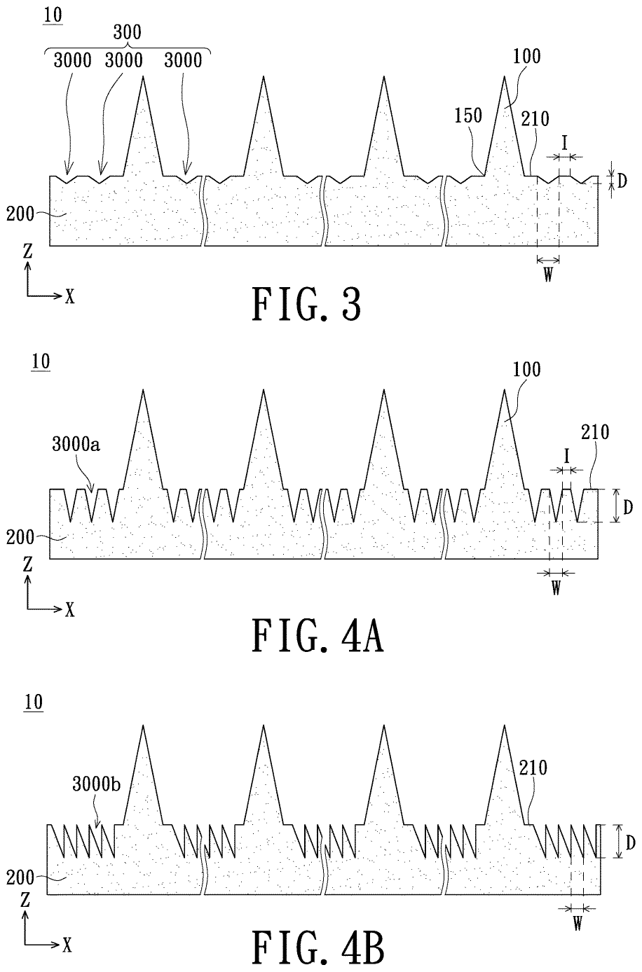

FIG. 2 is a partial schematic top view of the microneedle film 10 in FIG. 1. As shown in FIG. 2, the shape of the microneedles 100 is a quadra pyramid, though the invention is not limited thereto. The microneedle film 10 further includes a plurality of microstructures 300 formed on the first surface 210. The first surface 210 may have aforementioned roughness because of the microstructures 300. In some embodiments, the first surface 210 may have roughness between 0.05 μm and 0.1 μm. As shown in FIG. 2, the microstructures 300 may form a geometric shape, such as a circular shape, on the first surface 210, though the invention is not limited thereto. The microstructures 300 may form another geometric shape, such as a rectangle, a triangle, or other polygons. FIG. 3 is a partial cross-sectional schematic view of the microneedle film 10 taken along A′A′ in FIG. 2. As shown in FIG. 2 and FIG. 3, the microstructures 300 may be formed adjacent to the bottom 150 of each of the microneedles 100 on the first surface 210. Furthermore, multiple microstructures 300 are distributed between the adjacent microneedles 100 on the first surface 210.

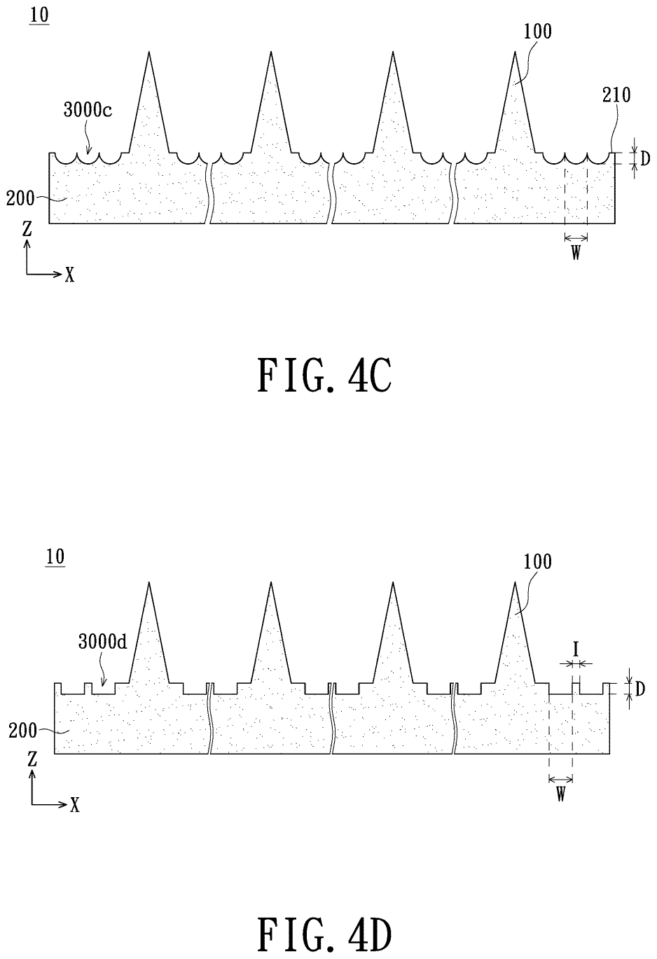

The microstructures 300 may further include a plurality of grooves 3000. In XY plane corresponding to the first surface 210, the grooves 300 are illustrated as dashed lines only for representation purpose while the grooves 3000 may be continuous and uninterrupted in practice. In addition, a quantity of the grooves 3000 in each of the microstructures 300 is not limited to the examples shown in the figures. The grooves 3000 of the microstructures 300 may have various shapes, which may vary depending on the mold used during the fabrication process of the microneedle film 10 (as described later). As shown in FIG. 3, the cross-sectional shape of the grooves 3000 in the microstructure 300 is an inverted triangle. However, the present invention is not limited thereto. FIG. 4A to FIG. 4D illustrate various shapes of the grooves 3000 of the microstructures 300. In the embodiment shown in FIG. 4A, each of the grooves 3000a (3000) is a serrated groove having an inverted triangular cross-section. In the embodiment shown in FIG. 4B, the grooves 3000b (3000) are serrated grooves distributed continuously. In the embodiment shown in FIG. 4C, each of the grooves 3000c (3000) has an arcuate cross-section with a smooth bottom. In the embodiment shown in FIG. 4D, each of the grooves 3000d (3000) has a rectangular cross-section with a flat bottom. In addition, the microstructures 300 of the microneedle film 10 are not limited to a single groove shape. For example, multiple shapes of the grooves 3000 may be disposed on the same microneedle film 10.

Furthermore, a mold used for manufacturing the microneedle film 100 may have a plurality microneedle structures and a plurality of micro-groove structures (as described later). The microneedle structures are used to form the microneedles 100, and the micro-groove structures are reflected on the first surface 210 of the base layer 200 to form the microstructures 300. The micro-groove structures of the mold may be formed, for example, by a cutting process. Each of the micro-groove structures includes a plurality of groove structures. The groove structures may have different cross-sections, such as those shown in FIG. 3 and FIG. 4A to FIG. 4D, depending on tools used in the cutting process. In addition, the grooves 3000 of the microstructures 300 may have various distribution (as described later). The micro-groove structures in the mold enhance the adhesion between the material of the microneedle film 10 and the mold, thereby improving the film formation quality of the microneedle film 10 and increasing the film formation yield.

The groove structures of the mold may have a width, for example, between 1 μm and 120 μm, and a depth, for example, between 1 μm and 10 μm, including 1 μm, 2 μm, 3 μm, 4 μm, 5 μm, 6 μm, 7 μm, 8 μm, 9 μm, and 10 μm. A spacing between the adjacent groove structures may be, for example, between 1 μm and 120 μm. Accordingly, as shown in FIG. 3 and FIG. 4A to FIG. 4D, when the groove structures of the mold are reflected onto the microneedle film 10, the grooves 3000 of the microstructures 300 have a width W approximately between 1 μm and 120 μm, and a depth D approximately between 1 μm and 10 μm, including 1 μm, 2 μm, 3 μm, 4 μm, 5 μm, 6 μm, 7 μm, 8 μm, 9 μm, and 10 μm. A spacing I between the adjacent grooves 3000 may be approximately between 0 μm and 120 μm. The width W is defined as the width of the groove 3000 perpendicular to its extending direction on the first surface 210. In the embodiment of the invention, due to the material properties of the microneedle film 10 and the volumetric shrinkage during molding, the width W of the grooves 3000 may be between 1 μm and 120 μm or less. For example, with a molding shrinkage of 1% to 2.5%, the width W may further range from 0.975 μm to 120 μm, where 0.975 μm corresponds to a 2.5% molding shrinkage from an intended width of 1 μm. For the same reason, the depth D may also range from 1 μm to 10 μm or less. For example, the depth D may shrink 1% to 2.5% and further range from 0.975 μm to 10 μm. The spacing I may also shrink 1% to 2.5% and further range 0.975 μm to 120 μm. In other words, the width W, the depth D, and the spacing I of the grooves 3000 are substantially the same as the width, the depth, and the spacing of the corresponding groove structures on the mold, except that they may be slightly reduced because of the material properties and shrinkage of the microneedle film 10 during molding. The molding shrinkage may be, for example, from 1% to 2.5%.

The width and the spacing of the groove structures may be coordinated with each other to allow the mold to achieve optimal adhesion to the material of the microneedle film 10. Accordingly, the width W and the spacing I of the grooves 3000 is also coordinated with each other. In some embodiments, when the width of the groove structures is between 1 μm and 10 μm, and the width W of the grooves 3000 is between 1 μm and 10 μm, the spacing between the adjacent groove structures is preferably between 1 μm and 10 μm, and the spacing I between the adjacent grooves 3000 is preferably between 0 μm and 10 μm. For example, when the width is, for example, 5 μm, the spacing I may be, for example, 0 μm, 1 μm, 2 μm, 3 μm, 4 μm, 5 μm, 6 μm, 7 μm, 8 μm, 9 μm, 10 μm, or a combination thereof. The invention is not limited thereto. It should be noted that due to the molding shrinkage, the width W may further range from 0.975 μm to 10 μm, and the spacing I may further range from 0.975 μm to 10 μm. When the width of the groove structures is between 10 μm and 30 μm, and the width W of the grooves 3000 is between 9.75 μm and 30 μm because of the molding shrinkage, the spacing between the adjacent groove structures is preferably between 10 μm and 30 μm, and the spacing I between the adjacent grooves 3000 is preferably between 9.75 μm and 30 μm. For example, when the width W is, for example, 20 μm, the spacing I may be, for example, 9.75 μm, 10 μm, 15 μm, 20 μm, 25 μm, 29.7 μm, 30 μm, or a combination thereof. The invention is not limited thereto. When the width of the groove structures is between 30 μm and 70 μm, and the width W of the grooves 3000 is between 29.25 μm and 70 μm because of the molding shrinkage, the spacing between the adjacent groove structures is preferably between 30 μm and 70 μm, and the spacing I between the adjacent grooves 3000 is preferably between 29.25 μm and 70 μm. For example, when the width W is, for example, 50 μm, the spacing I may be, for example, 29.25 μm, 30 μm, 35 μm, 40 μm, 45 μm, 50 μm, 55 μm, 60 μm, 65 μm, 69.3 μm, 70 μm, or a combination thereof. The invention is not limited thereto. When the width of the groove structures is between 70 μm and 120 μm, and the width W of the grooves 3000 is between 68.25 μm and 120 μm because of the molding shrinkage, the spacing between the adjacent groove structures is preferably between 70 μm and 120 μm, and the spacing I between the adjacent grooves 3000 is preferably between 68.25 μm and 120 μm. For example, when the width W is, for example, 100 μm, the spacing I may be, for example, 68.25 μm, 70 μm, 75 μm, 80 μm, 85 μm, 90 μm, 95 μm, 100 μm, 105 μm, 110 μm, 115 μm, 118.8 μm, 120 μm, or a combination thereof. The invention is not limited thereto. In any given microstructure 300, the spacing I between the adjacent grooves 3000 may be the same, or may be vary within the predetermined range described above.

As mentioned, the microstructures 300 may form a part of the first surface 210 that is adjoined or adjacent to the bottom 150 of each of the microneedles 100. Furthermore, the microstructures 300 on the first surface 210 may correspond to the microneedles 100, respectively. That is, each of the microneedles 100 corresponds to a respective one of the microstructures 300. The micro-groove structures in the mold, which is used to form the microstructures 300, may be formed through cutting tools, such as a milling cutter. The micro-groove structures may be distributed according to the microneedle structures in the mold. For example, the mill cutter may be used to form the micro-groove structures along the surrounding of each of the microneedle structures and centering on the microneedle structures, such that the micro-groove structures are adjoined or adjacent to the microneedle structures. When the micro-groove structures are formed close to the microneedle structures, they help ensure the adhesion between the mold and the material adjacent to the microneedles 100, thereby enhancing the molding of the microneedles 100. In some embodiments, the micro-groove structures enable the microneedles 100 of the microneedle film 10 to achieve a formation yield of 97.5% or more.

As shown in FIG. 2, the microstructures 300 are formed on the first surface 210 of the base layer 200, and the microstructures 300 correspond to the microneedles 100. In the embodiment shown in FIG. 2, each of the microstructures 300 corresponds to one respect of the microneedles 100 and includes the plurality of grooves 3000. Each of the grooves 3000 forms a geometric shape, such as a circular pattern. The circular patterns formed by the grooves 3000 may be distributed in a concentric configuration with the position of the microneedle 100 as the center. In the embodiment shown in FIG. 2, an area A of the microstructure 300 of the microneedle 100 on the first surface 210 is defined as an area covered from the innermost groove 3000 to the outermost groove 3000, and the area A has an edge E. As shown in FIG. 2, the microstructures 300 of the adjacent microneedles 100 may be in contact with each other, i.e., the edges E of adjacent microstructures 300 may be in contact, although the invention is not limited thereto. As shown in FIG. 2, the bottoms 150 of the adjacent microneedles 100 may be spaced apart by a first length L. In some embodiments of the invention, a distance between the edge E of the area A of the microstructure 300 of any microneedle 100 and the bottom 150 of the adjacent microneedle 100 may be less than half of the first length L, such that the edge E extends into the area A of the microstructure 300 of the adjacent microneedle 100.

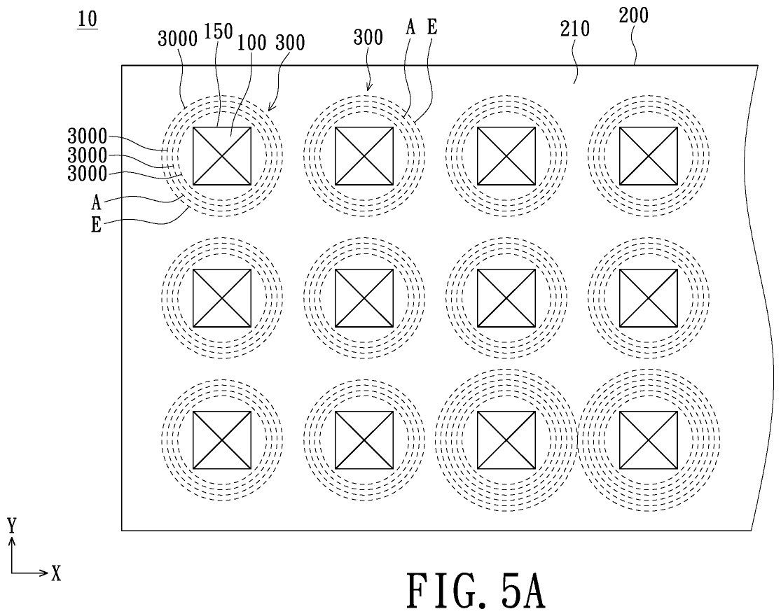

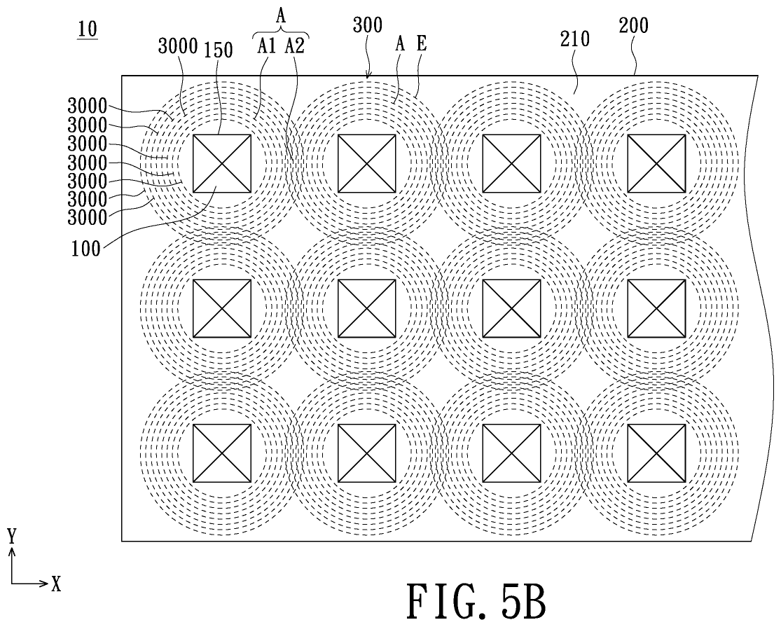

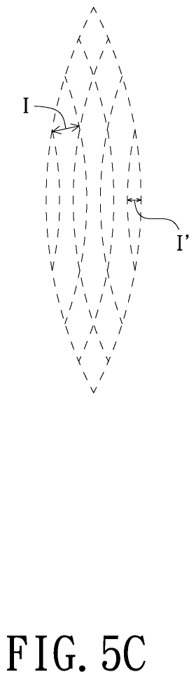

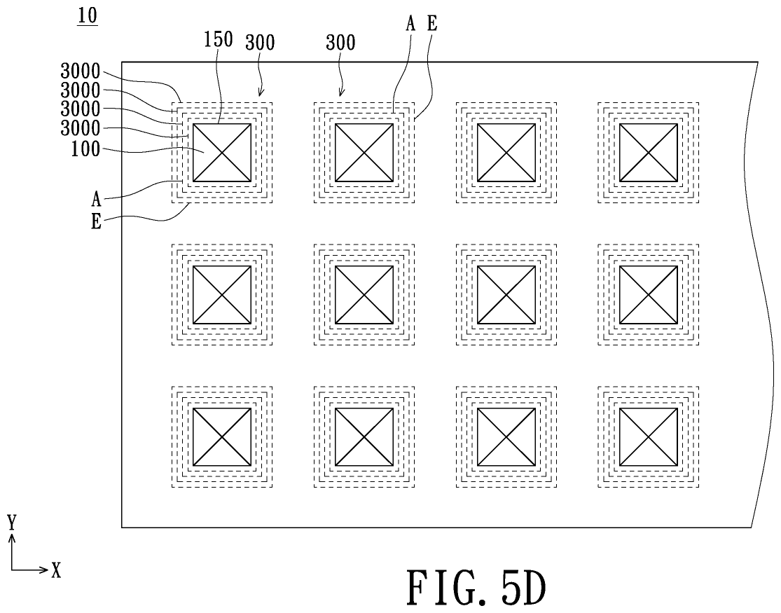

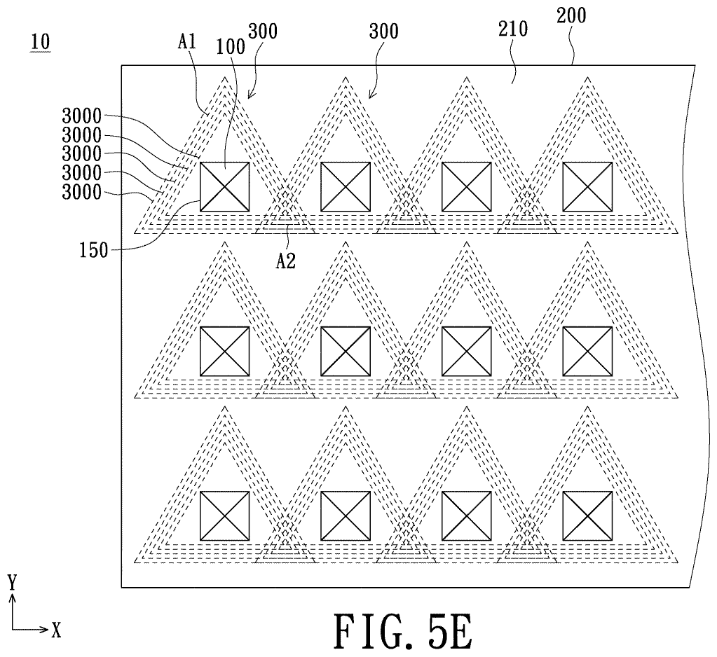

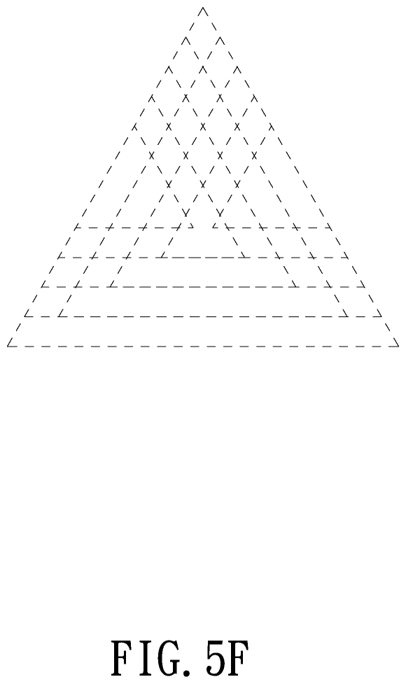

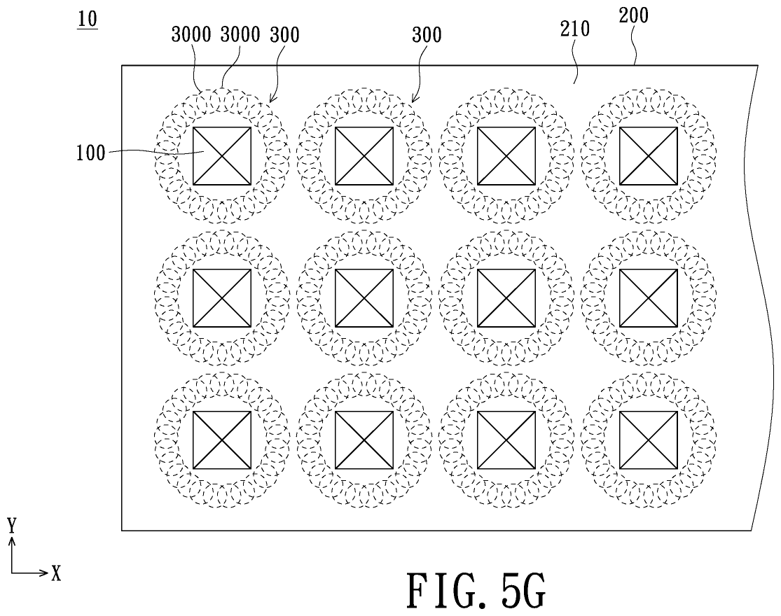

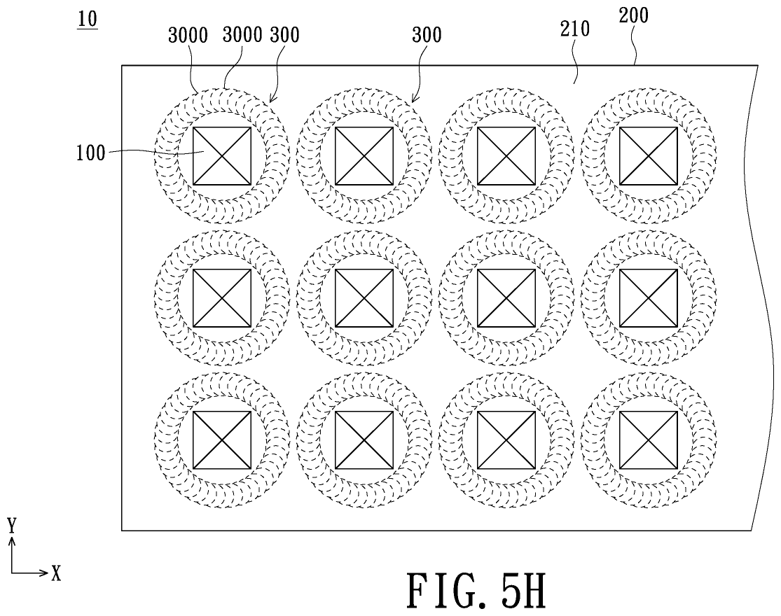

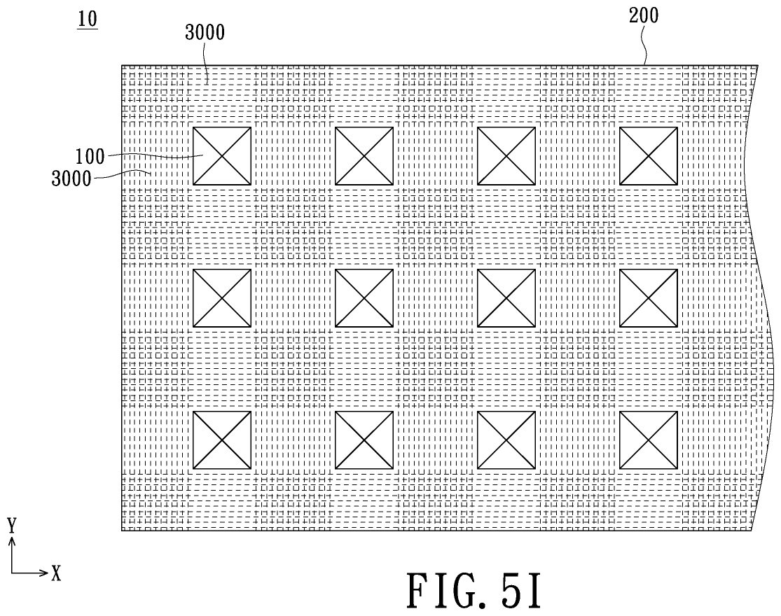

FIG. 5A and FIG. 5B are schematic diagrams illustrating the microstructures 300 with other configurations. As shown in FIG. 5A, the microstructures 300 of the adjacent microneedles 100 are spaced apart from each other. As shown in FIG. B, the microstructures 300 of the adjacent microneedles 100 overlap with each other. The aforementioned area A may further include a first area A1 and a second area A2. In the embodiment shown in FIG. 5B, preferably, the microstructures 300 do not overlap in the first area A1. Also, the width W of the grooves 3000 and the spacing I are configured as mentioned above. When the width W is between 0.975 μm and 10 μm, the spacing I is preferably between 0.975 μm and 10 μm. When the width W is between 9.75 μm and 30 μm, the spacing I is preferably between 9.75 μm and 30 μm. When the width W is between 68.25 μm and 120 μm, the spacing I is preferably between 68.25 μm and 120 μm. In the embodiment shown in FIG. 5B, the second area A2 has a crescent shape. In addition, the microstructures 300 corresponding to other microneedles 100 may appear in the second area A2. In other words, in the second area A2, partial overlapping may occur among the plurality of microstructures 300. Here, the partial overlapping includes intersection, superposition, or retention of only one of the overlapping portions. FIG. 5C is a partially enlarged schematic diagram of the second area A2 in FIG. 5B. As shown in FIG. 5C, when the microstructures 300 overlap with each other, a spacing I′ less than the spacing I may occur in the overlapping areas. For example, if the width W of the grooves 3000 of the microstructure 300 of each of the microneedles 100 is 10 μm and the corresponding spacing I is 10 μm, the spacing I′ less than 10 μm may be observed in the overlapping areas between microstructures 300 of the adjacent microneedles 100. However, the invention is not limited thereto. In other words, the grooves 3000 may be densely distributed in the overlapping portions. It should be noted that, in one embodiment, only the grooves 3000 of the single microstructure 300 is retained in the second area A2. Taking FIG. 5B and FIG. 5C as an example, each microstructure 300 is generally circular, as shown in FIG. 5B, and includes the grooves 3000. However, most of the microstructures 300, as shown on the right side in FIG. 5C, lack a crescent-shaped portion.

FIG. 5D and FIG. 5E are partial schematic top diagrams showing a microneedle film according other embodiments of the present invention. In comparison with the embodiment in FIG. 2, a major difference is that the embodiment in FIG. 5D includes the grooves 3000 which are arranged on the first surface 210 and form a plurality of similar rectangles. In comparison with the embodiment in FIG. 2, a major difference is that the embodiment in FIG. 5E includes the grooves 3000 which are arranged on the first surface 210 and form a plurality of similar triangles. Also, the microstructures 300 of the adjacent microneedles 100 overlap with each other. In the embodiment shown in FIG. 5E, the second area A2 of each microstructure 300 presents a smaller triangular shape. The second area A2 contains microstructures 300 from the adjacent microneedles 100. FIG. 5F is a partially enlarged schematic diagram showing the second area A2 in FIG. 5E. As shown in FIG. 5F, the grooves 3000 from the different microstructures 300 may intersect, overlap, remain non-interfering with one another, or be selectively retained. In case that the grooves 3000 overlap, the grooves 3000 may partially overlap and integrate, thereby producing the width W greater than the average. In some embodiments, the second area A2 shown in FIG. 5E may only contain the grooves of one of the microstructures 300. In other words, a small triangular region may be absent in most of the microstructures 300, such as the one on the right side of FIG. 5F.

FIG. 5G is a partial schematic top view of a microneedle film according to another embodiment of the present invention. In the embodiment shown in FIG. 5G, each of the grooves 3000 of the microstructures 300 also form a substantially circular shape. The difference between the embodiment in FIG. 5G and the embodiment in FIG. 2 is that the grooves 3000 are not distributed in a concentric pattern, but are sequentially disposed around the microneedle 100, such that the adjacent grooves 3000 partially overlap with each other. Due to the machining results generated by the cutting tool on the mold, in certain embodiments of the present invention, the grooves 3000 of each microstructure 300 reflected on the microneedle film 10 may only be partially retained. In other words, even though the cutting tool machines the mold by continuously drawing circles along the periphery of the microneedle structure to form the groove structures, and the circular machining paths partially overlap, the resulting microgroove structures and the corresponding microstructures 300 may be as shown in FIG. 5H. As shown in FIG. 5H, a portion (e.g., the right portion) of any groove structure and its corresponding groove 3000 may no longer be present due to subsequent machining of the groove structure located thereon.

FIG. 5H is a partial schematic top view of a microneedle film according to another embodiment of the present invention. In the embodiment shown in FIG. 5H, each of the grooves 3000 of the microstructures 300 forms a curved line segment, and the curved line segments are arranged around the microneedles 100. In some embodiment, the curved line segment may be an arc line segment. It is noted that FIG. 2 and FIG. 5A to FIG. 5H are merely illustrative, and the blank areas on the first surface 210 do not necessarily indicate the absence of the microstructures 300. In addition, the microstructures 300 may be fully distributed over the first surface 210. The portion of the first surface 210 where the microstructures 300 are distributed may have a surface roughness (Ra) of, for example, 0.05 μm to 0.1 μm. For example, the portion where the microstructures 300 are distributed may have a surface roughness of 0.05 μm, 0.06 μm, 0.07 μm, 0.08 μm, 0.09 μm, or 0.1 μm, or a combination thereof.

FIG. 5I is a partial schematic top view of a microneedle film according to another embodiment of the present invention. During the fabrication of the mold, the micro-groove structures may be formed according to the microneedle structures (e.g., formed around the microneedle structures as centers), or may alternatively be form by other methods. For example, in some embodiments, the milling cutter may be used to form the micro-groove structures along a longitudinal direction and a transverse direction on the mold, such that the microgroove structures are distributed over the mold. As reflected on the microneedle film 10 in an embodiment shown in FIG. 5I, the grooves 3000 of the microstructures 300 appear as straight lines extending in the longitudinal direction (Y-direction) and in the transverse direction (X-direction) on the first surface 210. The longitudinal grooves 3000 and the transverse grooves 3000 may surround each of the microneedles 100. In other words, the microstructure 300 corresponding to the respective microneedles 100 is formed by the plurality of longitudinal grooves 3000 and transverse grooves 3000. The longitudinal grooves 3000 and the transverse grooves 3000 may intersect or only one of them may be retained. For example, for the adjacent microneedles 100 in the Y-direction, the respective longitudinal grooves 3000 may be connected. For the adjacent microneedles 100 in the X-direction, the respective transverse grooves 3000 may be connected. It should be understood that FIG. 5A to FIG. 5I are merely illustrative, and the microstructures 300 of the embodiments of the present invention may have various distribution configurations.

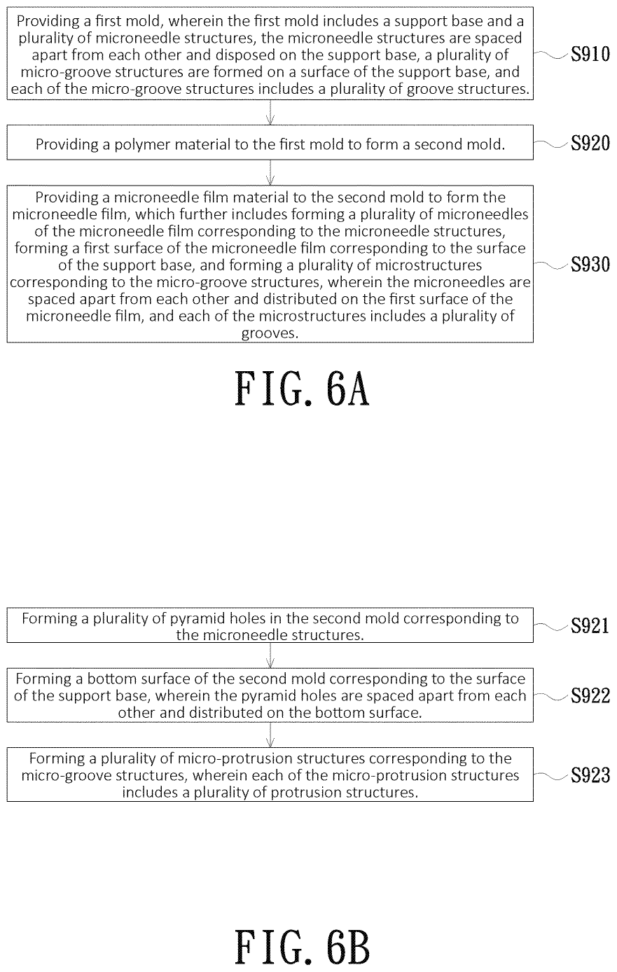

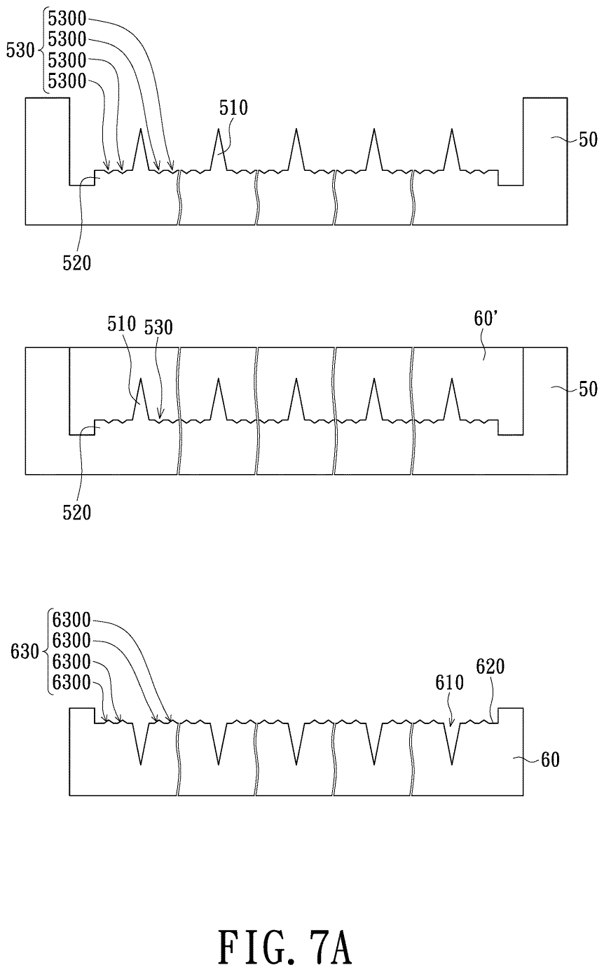

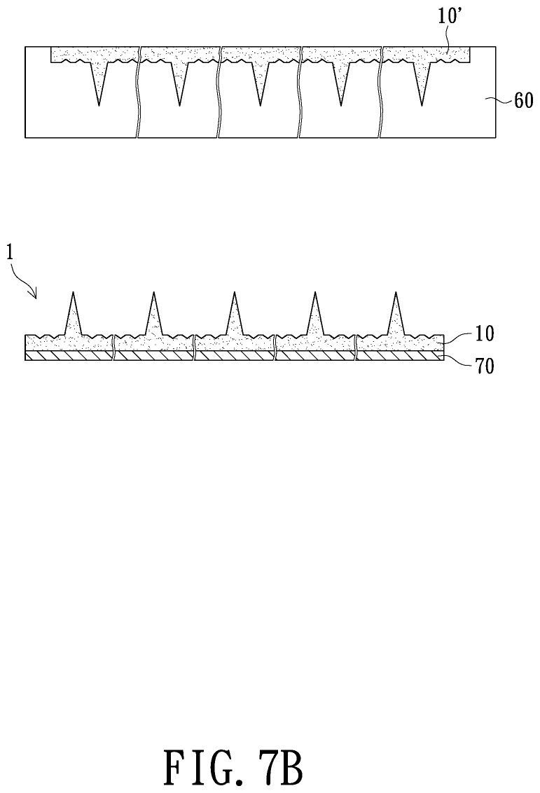

The invention further provides a method of manufacturing a microneedle film. In the embodiment of the invention, as shown in FIG. 6A, the method of manufacturing the microneedle film includes providing a first mold (Step S910). The first mold includes a support base and a plurality of microneedle structures. A plurality of micro-groove structures are formed on a surface of the support base, and each of the micro-groove structures corresponds to a respective one of the microneedle structures. Each of the micro-groove structures includes a plurality of groove structures. Next, a polymer material is provided to the first mold to form a second mold (Step S920). After that, a microneedle film material is provided to the second mold to form the microneedle film (Step S930). Step S930 further includes forming a plurality of microneedles corresponding to the microneedle structures, forming a first surface of the microneedle film corresponding to the surface of the support base, and forming a plurality of microstructures corresponding to the micro-groove structures. The microneedles are spaced apart from each other and distributed on the first surface. The microstructures includes a plurality of grooves. FIG. 7A and FIG. 7B are schematic diagrams showing the aforementioned manufacturing process according to Steps S910 to S930.

As shown in FIG. 7A, a first mold 50 is first provided. The first mold 50 may be a male mold and preferably a metal mold. The material of the first mold 50, for example, may be titanium, copper, aluminum, nickel, tungsten, stainless steel, titanium alloy, nickel alloy, aluminum alloy, or copper alloy, but is not limited thereto. As shown in FIG. 7A, a plurality of microneedle structures 510 are spaced apart from each other and are disposed on a support base 520 with an appropriate distribution density. The spacing between the adjacent microneedle structures 510 may be, for example, from 0.5 mm to 5 mm. Each of the microneedle structures 510 may have a tapered shape with a wide bottom and a narrow top, such as a conical, triangular pyramidal, or quadrangular pyramidal shape. The height of the microneedle structures 510 may be, for example, from 150 μm to 1000 μm.

The micro-groove structures 530 on the surface of the support base 520 may be formed via machining, such as a cutting process. More specifically, the micro-groove structures 530 may be formed by a milling process. Each of the micro-groove structures 530 includes a plurality of groove structures 5300. The groove structures 5300 may be formed by a machining tool such as a milling cutter. Different milling cutters may form groove structures 5300 of different shapes, for example, having a triangular, arcuate, or rectangular cross-sectional profile. The groove structures 5300 may have a width of, for example, 1 μm to 120 μm, and a depth of, for example, 1 μm to 10 μm. The spacing between the adjacent groove structures 5300 may be, for example, 0 to 120 μm. Through the action of the machining tool, the groove structures 5300 may further form geometric patterns or line segments on the surface of the support base 520 (referring to FIG. 2 and FIG. 5A to FIG. 5H). In addition, the machining tool may form the micro-groove structures 530 concentrically around each microneedle structure 510, but is not limited thereto. The micro-groove structures 530 corresponding to adjacent microneedle structures 510 may be connected, overlapped, or separated from each other.

As shown in FIG. 7A, Step S920 includes providing a polymer material 60′ to the support base 520 and the microneedle structures 510 and micro-groove structures 530 disposed thereon. The polymer material 60′ may be, for example, polyethylene, polypropylene, polylactic acid, polybutylene succinate, polydimethylsiloxane, but is not limited thereto. As shown in FIG. 6B, Step S920 further includes forming a plurality of conical holes in a second mold corresponding to the plurality of microneedle structures (Step S921), forming a bottom surface in the second mold corresponding to the surface of the supporting base (Step S922), and forming a plurality of micro-protrusion structures corresponding to the plurality of microgroove structures (Step S923). Each of the micro-protrusion structures includes a plurality of protrusion structures. The second mold 60 may be a female mold. As shown in FIG. 7A, the plurality of conical holes 610 in the second mold 60 are formed by the microneedle structures 510 of the first mold 50 through the polymer material 60′. The plurality of micro-protrusion structures 630 are formed by the microgroove structures 530 of the first mold 50 through the polymer material 60′. Each of the micro-protrusion structures 630 includes a plurality of protrusion structures 6300. Each of the protrusion structures 6300 may have a width, for example, between 1 μm and 120 μm, and a height, for example, between 1 μm and 10 μm. A spacing between the adjacent protrusion structures 6300 may be, for example, 0 to 120 μm. The portion of the polymer material 60′ in contact with the surface of the support base 520 is molded into the bottom surface 620 of the second mold 60.

As shown in FIG. 7B, Step 930 includes further providing the microneedle film material 10′ to the conical holes 610, and allowing the microneedle material 10′ to be distributed over the bottom surface 620 of the second mold 60 and the micro-protrusion structures 630 on the bottom surface 620. The microneedle film material 10′ may include, for example, maltose, sucrose, trehalose, lactose, dextrin, maltodextrin, β-cyclodextrin, 2-hydroxypropyl-β-cyclodextrin, dextran, amylopectin, sodium hyaluronate, methyl vinyl ether-maleic anhydride copolymer, sodium carboxymethyl cellulose, methyl cellulose, hydroxypropyl methyl cellulose, hydroxypropyl cellulose, gelatin, polyvinyl alcohol, polyvinylpyrrolidone, polyethylene glycol, polylactic acid, polyglycolic acid, polylactic-co-glycolic acid, chitosan, a combination thereof, or any other biocompatible material suitable for transdermal drug delivery. Step S930 may further include drying the microneedle film material 10′ to form the microneedle film 10.

The micro-protrusion structures 630 increase the contact area between the second mold 60 and the microneedle film material 10′, and more particularly, the surface having the conical holes 610 and the microneedle film material 10′. Furthermore, during the process of forming the microneedle film material 10′ into the microneedle film 10, the micro-protrusion structures 630 can enhance the adhesion of the bottom surface 620 to the microneedle film material 10′, thereby facilitating the retention of the microneedle film material 10′ on the second mold 60 and ensuring that the microneedle film material 10′ does not easily detach from the second mold 60 before the molding process is completed. In some embodiments, the adhesion force between the bottom surface 620 and the microneedle film 10 can be inferred by measuring the ease with which the microneedle film 10 can be peeled from the bottom surface 620. A greater required peeling force indicates a stronger adhesion provided by the bottom surface 620 and the micro-protrusion structures 630 to the microneedle film 10. In comparison with conventional male molds without the micro-groove structures and their corresponding female molds without the micro-protrusion structures, the second mold 60 of the present invention can increase the peeling force by more than two times. Furthermore, in the embodiment of the invention, the peeling force can be further enhanced by varying the width, spacing, and a combination thereof of the protrusion structures 6300. For example, when the protrusion structures 6300 have a width of 30 μm to 70 μm and a spacing of 30 μm to 70 μm between adjacent protrusion structures 6300, the peeling force can be increased by two times. When the width is 1 μm to 10 μm and the spacing is also 1 μm to 10 μm, the peeling force can be increased by more than two times. It is understood that the relationship between the width and spacing of the protrusion structures 6300 and the peeling force may vary depending on the specific microneedle film material 10′ used. In other words, the width and spacing of the protrusion structures 6300 may be adjusted according to the characteristics of the microneedle film material 10′ in order to achieve an optimal adhesion force. The following example illustrates the measured peeling forces under a 65 mm×45 mm area using different combinations of widths and spacings of the protrusion structures 6300. Here, the initial peeling point is fixed to a force gauge, and a peeling force is applied at an angle of 90 degrees thereto.

| | | | Peeling | | |
|---|---|---|---|---|---|
| | Width | Spacing | force | Degree of | |
| | (μm) | (μm) | (g) | increase | Result |
| Control group | 0 | 0 | 32 | 1 | NG |
| (no microstructure) | | | | | |
| Experimental | 1 to 10 | 1~10 | 73 | 2.28 | OK |
| group 1 | | | | | |
| Experimental | 10 to 30 | 10~30 | 66 | 2.03 | OK |
| group 2 | | | | | |
| Experimental | 30 to 70 | 30~70 | 64 | 2.00 | OK |
| group 3 | | | | | |
| Experimental | 70 to 120 | 70~120 | 45 | 1.41 | OK |
| group 4 | | | | | |

When the microneedle film material 10′ is dried and formed into the microneedle film 10, a portion of the microneedle film material 10′ located within the conical holes 610 may be formed into the microneedles 100 of the microneedle film 10, a portion of the microneedle film material 10′ in contact with the bottom surface 620 may be formed into the first surface 210, and a portion of the microneedle film material 10′ disposed on the micro-protrusion structures 630 may be formed into the microstructures 300 on the first surface 210. Since the groove structures 5300 and the corresponding protrusion structures 6300 have, for example, a width approximately between 1 μm and 120 μm, a depth approximately between 1 μm and 10 μm, and a spacing approximately between 0 μm and 120 μm, the grooves 3000 formed from the protrusion structures 6300 may have a width W approximately between 1 μm and 120 μm, a depth D approximately between 1 μm and 10 μm, and a spacing I approximately between 0 μm and 120 μm. Due to the material properties of the microneedle film material 10′, as well as its shrinkage behavior during molding, the microneedle film 10 may slightly shrink. Specifically, the grooves 3000 formed therein may exhibit a molding shrinkage of approximately 1% to 2.5% in terms of width W, depth D, and spacing I relative to the dimensions of the groove structures 5300 of the first mold 50. For example, when the groove structures 5300 of the first mold 50 have the width of 100 μm and the depth of 5 μm, and the spacing between the adjacent groove structures 5300 is 100 μm, the corresponding grooves 3000 may have the width W between 97.5 μm and 100 μm, such as 97.5 μm, 98 μm, 98.5 μm, 99 μm, 99.5 μm, or 100 μm, the depth D between 4.875 μm and 5 μm, such as, 4.875 μm, 4.9 μm, 4.95 μm, or 5 μm, and the spacing I between the adjacent grooves 3000 between 97.5 μm and 100 μm, such as 97.5 μm, 98 μm, 98.5 μm, 99 μm, 99.5 μm, or 100 μm. However, the invention is not limited thereto.

As shown in FIG. 7B, after the microneedle film 10 is formed, a carrier layer 70 may be further disposed on the bottom surface thereof, i.e., the second surface 220, and demolding may be performed to form the microneedle patch 1. In summary, the microneedle film 10 and the microneedle patch 1 according to the embodiment of the present invention exhibit a high degree of molding completeness. Accordingly, the film formation quality is improved, the microneedle structures 100 are structurally intact and dimensionally accurate, thereby improving the production yield.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle film, comprising:
a base layer, comprising a first surface and a second surface opposite to the first surface, the first surface being rougher than the second surface;
a plurality of microneedles, disposed on the base layer, the microneedles being spaced apart from each other and distributed on the first surface of the base layer; and
a plurality of microstructures, formed on the first surface, each of the microstructures being corresponding to a respective one of the microneedles;
wherein each of the microstructures comprises a plurality of grooves, each of the grooves forms a geometric shape or a line segment on the first surface, and the line segment comprises a straight line or a curved line.

2. The microneedle film according to claim 1, wherein each of the grooves has a width between 0.975 μm and 120 μm and a depth between 0.975 μm and 10 μm, and a spacing between the two adjacent grooves is between 0 μm and 120 μm.

3. The microneedle film according to claim 2, wherein the width of the groove is between 0.975 μm and 10 μm, and the spacing between the two adjacent grooves is between 0 μm and 10 μm.

4. The microneedle film according to claim 2, wherein the width of the groove is between 9.75 μm and 30 μm, and the spacing between the two adjacent grooves is between 9.75 μm and 30 μm.

5. The microneedle film according to claim 2, wherein the width of the groove is between 29.25 μm and 70 μm, and the spacing between the two adjacent grooves is between 29.25 μm and 70 μm.

6. The microneedle film according to claim 2, wherein the width of the groove is between 68.25 μm and 120 μm, and the spacing between the two adjacent grooves is between 68.25 μm and 120 μm.

7. The microneedle film according to claim 1, wherein at least a portion of the first surface has a surface roughness ranging from 0.05 μm to 0.1 μm, and the microstructures adjacent to the microneedles are in contact with each other, overlap with each other, or are separated from each other.

8. The microneedle film according to claim 1, wherein the geometric shape comprises a circular shape, each of the microstructures comprises a plurality of the circles formed of the grooves on the first surface, and the circles are concentrically arranged with respect to a position of the corresponding microneedle.

9. The microneedle film according to claim 1, wherein the curve line comprises a curve section, each of the microstructures comprises a plurality of the curve sections formed of the grooves on the first surface, and the curve sections are arranged surrounding the corresponding microneedle.

10. The microneedle film according to claim 1, wherein the geometric shape comprises a rectangular shape, each of the microstructures further comprises a plurality of similar rectangular shapes formed of the grooves on the first surface, and the similar rectangular shapes are concentrically arranged with respect to a position of the corresponding microneedle.

11. The microneedle film according to claim 1, wherein the geometric shape comprises a triangular shape, each of the microstructures further comprises a plurality of similar triangular shapes formed of the grooves on the first surface, and the similar triangular shapes are concentrically arranged with respect to a position of the corresponding microneedle.

12. The microneedle film according to claim 1, wherein the microstructure corresponding to each of the microneedles defines a first region on the first surface, each of the grooves within the first region has a width between 0.975 μm and 120 μm, and a spacing between the adjacent grooves within the first region is between 0 μm and 120 μm.

13. The microneedle film according to claim 12, wherein the microstructure corresponding to each of the microneedles further defines a second region on the first surface, and another microstructure corresponding to another microneedle adjacent to the microneedle is further distributed within the second region.

14. A method of manufacturing the microneedle film according to claim 1, the method comprising:
providing a first mold, the first mold comprising a support base and a plurality of microneedle structures, the microneedle structures being spaced apart from each other and disposed on the support base, the support base comprising a plurality of micro-groove structures formed on a surface of the support base, the micro-groove structures being corresponding to the microneedle structures respectively, each of the micro-groove structures comprising a plurality of groove structures;
providing a polymer material to the first mold to form a second mold; and
providing a microneedle film material to the second mold to form the microneedle film, which comprises:
forming the microneedles of the microneedle film corresponding to the microneedle structures;
forming the first surface of the microneedle film corresponding to the surface of the support base, the microneedles being spaced apart from each other and distributed on the first surface; and
forming the microstructures corresponding to the micro-groove structures, each of the microstructures comprising the grooves.

15. The method according to claim 14, wherein each of the groove structures further forms a geometric shape or a line segment on the surface of the support base, and the micro-groove structures corresponding to the adjacent microneedle structures are in contact with each other, overlap with each other, or are separated from each other.

16. The method according to claim 14, wherein the groove structure has a width between 1 μm and 120 μm and a depth between 1 μm and 10 μm, and a spacing between the adjacent groove structures is between 0 μm and 120 μm.

17. The method according to claim 16, wherein the width of the groove structure is between 1 μm and 10 μm, and the spacing between the adjacent groove structures is between 0 μm and 10 μm.

18. The method according to claim 16, wherein the width of the groove structure is between 10 μm and 30 μm, and the spacing between the adjacent groove structures is between 10 μm and 30 μm.

19. The method according to claim 16, wherein the width of the groove structure is between 30 μm and 70 μm, and the spacing between the adjacent groove structures is between 30 μm and 70 μm.

20. The method according to claim 16, wherein the width of the groove is between 70 μm and 120 μm, and the spacing between the adjacent grooves is between 70 μm and 120 μm.

21. The method according to claim 14, wherein forming the second mold further comprises:
forming a plurality of pyramid holes in the second mold corresponding to the microneedle structures;
forming a bottom surface of the second mold corresponding to the surface of support base, the pyramid holes being spaced apart from each other and distributed on the bottom surface; and
forming a plurality of micro-protrusion structures corresponding to the micro-groove structures, each of the micro-protrusion structures comprising a plurality of protrusion structures.
